(19) United States
(12) Patent Application Publication
(10) Pub. No.: US 2007/0112275 A1
(43) Pub. Date: May 17, 2007

(54) Medical Intervention Indicator Methods and Systems

(76) Inventors: William H. Cooke, San Antonio, TX (US); John B. Holcomb, Fort Sam Houston, TX (US); Jose Salinas, Fort Sam Houston, TX (US); Victor A. Convertino, Fort Sam Houston, TX (US)

Correspondence Address: OFFICE OF THE STAFF JUDGE ADVOCATE;U.S. ARMY MEDICAL RESEARCH AND MATERIEL COMMAND, ATTN: MCMR-JA (MS. ELIZABETH ARWINE), 504 SCOTT STREET, FORT DETRICK, MD, 21702-5012, US

(21) Appl. No.: 11/464,826

(22) Filed: Aug. 15, 2006

Related U.S. Application Data
(60) Provisional application No. 60/707,955, filed on Aug. 15, 2005.
(60) Provisional application No. 60/822,212, filed on Aug. 11, 2006.

(51) Int. Cl. A61B 5/04

(52) U.S. Cl. 600/513

(57) ABSTRACT

An approach for improving the chances of survival of an individual who has received a trauma including, for example, hemorrhage or blunt injury, by providing more relevant information regarding the individual to first responders including at least one of heart rate variability index value, a baroreflex sensitivity value, and a pulse pressure. This information being used in at least one implementation to provide medical treatment to injured individuals including dispatching assistance and/or prioritizing in a triage situation increasing the speed at which these decisions can be made. In one exemplary embodiment, the heart rate variability index value is determined based on the relative power of the high frequencies versus the relative power of the low frequencies. In one exemplary embodiment, the pulse pressure is determined based on the difference between systolic pressure and diastolic pressure.

This patent application claims the benefit of U.S. Provisional Application Ser. No. 60/707,955 filed Aug. 15, 2005 and entitled “Heart Rate Variability, Baroreflex Sensitivity, and Pulse Pressure to Predict Hemorrhage Severity,” and U.S. Provisional Application Ser. No. 60/822,212 filed Aug. 11, 2006 and entitled “Remote Triage and Monitoring System and Method,” which are hereby incorporated by reference.

### I. FIELD OF THE INVENTION

This invention relates to use of an indicator based at least on one of heart rate variability, baroreflex sensitivity, and pulse pressure to determine when medical intervention is required, for example, in a trauma situation. In further exemplary embodiments, using the indicator in a system and method for remote determination of whether an individual requires medical attention.

### II. BACKGROUND OF THE INVENTION

Acute uncontrolled hemorrhage, subsequent circulatory collapse, and resulting shock account for about 50% of the deaths on the battlefield and up to 82% of the early operative deaths from trauma in the civilian arena. However, once the trauma patient arrives at the hospital with hemostasis obtained and resuscitation completed, the mortality rate from hemorrhage drops to between 2% and 4%. Therefore, it is likely that the survival rate from severe hemorrhage may be improved, particularly in mass casualty or remote situations, by enhancing the capabilities for early, more accurate diagnosis, improved triage decision support to first level responders, and effective interventions.

The vital sign monitors placed in emergency transport vehicles provide the medic with routine measures of arterial systolic, diastolic and mean blood pressures, heart rate, and arterial oxygen carrying capacity (SpO₂) of trauma patients. Abnormalities in these vital signs, particularly in the presence of poor motor scores, can provide medics with excellent decision-support information regarding triage categories, evacuation priority, and required interventions. Unfortunately, such abnormalities are late predictors of poor outcomes because of compensatory mechanisms that buffer against changes in arterial blood pressure and SpO₂. Mortality from hemorrhage could be reduced with identification of other noninvasive hemodynamic measurements that provide early assessment of circulatory shock.

Currently, first responders (paramedics or combat medics) measure heart rate and blood pressure primarily as indicators of injury severity. However, measures of heart rate and blood pressure provide no indication as to the amount of blood a bleeding patient or soldier is losing as a function of time.

Manual vital sign assessment of traumatically-injured patients fails to provide early indications of physiological decompensation when the systolic blood pressure (SBP) is greater than 90 mmHg and the motor component of the Glasgow Coma Score (mGCS) equals 6, and is dependent on the first responder having physical access to the patient. Initial compensations to traumatic injury are driven importantly by autonomic neural regulation, but first responders have no tools to assess autonomic function directly. Previous studies have shown that elevated parasympathetic neural activity is associated with mortality in head trauma patients in an intensive care unit. Winchell, R J, “Spectral Analysis of Heart Rate Variability in the ICU: A Measure of Autonomic Function,” Journal of Surgical Research, 1996, 63:11-16; Winchell, R J et al., “Analysis of Heart-rate Variability: A Noninvasive Predictor of Death and Poor Outcome in Patients with Severe Head Injury,” Journal of Trauma, 1997, 43:927-933; and Baillard, C., “Brain Death Assessment Using Instant Spectral Analysis of Heart Rate Variability,” Critical Care Medicine, 2002, 30:306-310.

A trauma patient presenting with a systolic blood pressure of 90 or less mmHg usually requires rapid diagnosis and intervention. Bleeding patients with blood pressures greater than 90 mmHg can progress quickly toward cardiovascular collapse and shock because blood pressure before cardiovascular collapse does not accurately track blood loss; however, because the blood pressure indicates the patient is alright they may not receive the needed medical attention to prevent the cardiovascular collapse. Stroke volume reflects central volume directly, but stroke volume cannot be obtained easily by a first responder or early in the emergency department.

Currently, vital signs used for patient diagnosis and triage in both the prehospital and hospital settings do not accurately represent the injury severity of trauma patients. This is due to the inherent compensatory physiologic mechanisms that mask the true patient status until the patient approaches physiologic exhaustion.

Currently, over and under triage of trauma patients is a critical issue in both the civilian and military environments. Misclassified patients that are transported to inappropriate care sites result in higher mortality rates and/or increase in cost for treating patients in trauma centers when trauma care was not required. This problem is partly due to the inability of currently measured vital signs to accurately determine the actual injury severity of a trauma patient.

There is currently no device capable of estimating noninvasively changes in blood volume during hemorrhage. There is currently no device capable of providing the first responder with information necessary to predict the onset of hemorrhagic shock and death.

### III. SUMMARY OF THE INVENTION

In at least one exemplary embodiment according to the invention, a system will return real-time values for heart rate variability, autonomic balance, baroreflex sensitivity, and pulse pressure. Heart rate variability, autonomic balance, baroreflex sensitivity, and pulse pressure are different in patients who eventually die and change predictably in research subjects submitted to a simulated hemorrhage. The primary advantage of tracking estimated changes in blood loss rather than arterial pressure and heart rate is that the first responder will have advanced warning that a patient may be progressing toward hemorrhagic shock. Such advantages will help save the lives of both trauma victims and casualties of war.

At least one exemplary embodiment according to the invention can be used in remote monitoring of individuals without the need for invasive sensors using existing wireless infrastructures. Additionally, the ability to accurately determine the individual's status remotely provides the user with a remote triage capability that can be used in both the civilian and military environment to accurately classify groups of trauma patients and prioritize the evacuation and/or transport destinations of each patient.

At least one exemplary embodiment according to the invention uses currently available vital sign measurements to compute at least one new vital sign selected from heart rate variability, pulse pressure, and shock index to provide an early indication of cardiovascular collapse and thus the actual patient status to provide better and more accurate triage and treatments. By providing earlier indicators of the patient's status, field triage may be more accurate and help to reduce misclassifications of patients and improve patient outcomes and reduce overtriage situations.

At least one exemplary embodiment according to the invention includes a method comprising receiving an electrocardiogram from at least one remote individual, detecting R-waves within the electrocardiogram, calculating R-R interval power spectra for the electrocardiogram, calculating power spectral densities for a range of low frequencies and a range of high frequencies for the electrocardiogram, calculating an index based on the power spectral density of the range of high frequencies divided by the power spectral density of the range of low frequencies, and outputting the index.

At least one exemplary embodiment according to the invention includes a method comprising receiving systolic arterial pressure and diastolic arterial pressure from at least one individual, obtaining a pulse pressure based on the received systolic arterial pressure and the received diastolic arterial pressure, and outputting the pulse pressure.

At least one exemplary embodiment according to the invention includes a method comprising: receiving vital sign information including arterial pressures and electrocardiogram from a plurality of remote individuals; obtaining a pulse pressure for each individual; detecting R-waves in received electrocardiograms; determining R-R intervals in the electrocardiogram for each individual; when three or more sampling periods for an individual the systolic pressure is progressively increasing or decreasing systolic pressures and the R-R intervals are progressively increasing or decreasing, calculating a baroreflex sensitivity; for each individual performing a fast Fourier transform on the received electrocardiogram to obtain R-R interval power spectra, calculating power spectral densities for a range of low frequencies and a range of high frequencies, calculating an index equal to the power spectral density of the range of high frequencies divided by the power spectral density of the range of low frequencies; and notifying an entity when at least one indicator selected from a group consisting of the index exceeding a predetermined value, the pulse pressure is lower than a predetermined value, and the baroreflex sensitivity is trending lower.

Given the following enabling description of the drawings, the apparatus should become evident to a person of ordinary skill in the art.

### IV. BRIEF DESCRIPTION OF THE DRAWINGS

The present invention is described with reference to the accompanying drawings. In the drawings, like reference numbers indicate identical or functionally similar elements.

FIG. 1 illustrates an exemplary method for determining an index for an individual according to the invention.

FIG. 2 illustrates exemplary R-R intervals in the time domain.

FIG. 3 illustrates an example of heart rate variability analysis according to the invention.

FIG. 4A and 4B illustrate how patients who die have higher parasympathetic predominance and lower sympathetic predominance as estimated from calculations of autonomic balance using Fourier spectral analysis according to at least one exemplary embodiment of the invention.

FIG. 5 illustrates mean arterial pressure, baroreflex sensitivity, and heart rate variability (high frequencies) as a function of increasing lower body negative pressure used to simulate a hemorrhage.

FIG. 6 illustrates an exemplary method for determining the pulse pressure of an individual according to the invention.

FIG. 7 illustrates mean arterial pressure and pulse pressure as a function of increasing lower body negative pressure used to simulate a hemorrhage.

FIG. 8 illustrates systolic pressure, diastolic pressure, mean arterial pressure and pulse pressure for trauma patients who died and lived.

FIG. 9 illustrates an exemplary method for determining heart rate variability according to the invention.

FIG. 10 illustrates an exemplary method for determining a shock index according to the invention.

FIG. 11 illustrates an exemplary method for monitoring at least one individual according to the invention.

FIG. 12 illustrates a conceptual block diagram according to at least one exemplary embodiment of the invention.

FIG. 13 illustrates a conceptual block diagram according to at least one exemplary embodiment of the invention.

FIG. 14 illustrates an exemplary display screen according to the invention.

FIG. 15 illustrates ECG data, R-R interval data as a function of time, and R-R interval spectral power as a function of frequency as discussed in Example 2.

FIG. 16 illustrates a graphical representation of all of the survivors versus all of the non-survivors with an estimated normal range superimposed as discussed in Example 2.

FIG. 17 illustrates two examples of R-R interval spectral power, the top graph is of a patient who survived and had an index of 0.24 and the bottom graph is of a patient who died and had an index of 3.3 as discussed in Example 2.

FIG. 18 shows original tracings of arterial pressures and ECG along with systolic arterial pressure and R-R interval as a function of time as discussed in Example 3.

FIG. 19 shows R-R intervals and associated frequency domain representations for one representative subject during baseline (0 mmHg), −60 mmHg decompression, and return to 0 mmHg as discussed in Example 3.

FIG. 20 shows how LBNP caused progressive decreases in both heart rate variability and cardiac baroreflex sensitivity of both up and down baroreflex sequences, while arterial pressure remained largely unchanged as discussed in Example 3.

FIG. 21 shows characteristics of baroreflex gains across time as discussed in Example 3.

FIG. 22 shows the relationship between progressive increases in LBNP and the mean (±standard error) for mean arterial pressure, stroke volume, pulse pressure, and MSNA as discussed in Example 4.

### V. DETAILED DESCRIPTION OF THE DRAWINGS

The invention is directed at approaches for use in trauma and/or triage situations to provide additional information to the first responders to increase the odds of a successful outcome by indicating when a medical intervention is required prior to current approaches. In other exemplary embodiments according to the invention, the system and method provide for remote monitoring of individuals so that medical care can be provided if indicated as being needed. Remote is used to indicate that the individual being monitored is not observable in the field of vision by the person or device monitoring the individual. The invention in at least one exemplary embodiment includes a device and method capable of calculating in real time at least one of heart rate variability, baroreflex sensitivity, and pulse pressure in bleeding patients as a tool to detect magnitudes of blood loss and gain more timely information regarding the patient's condition. The calculation of heart rate variability produces a ratio of the high frequency power versus the low frequency power. The baroreflex sensitivity over time can provide a trend indication as to whether an individual is approaching a cardiovascular collapse. The calculation of the pulse pressure is based on the difference between the systolic pressure and the diastolic pressure.

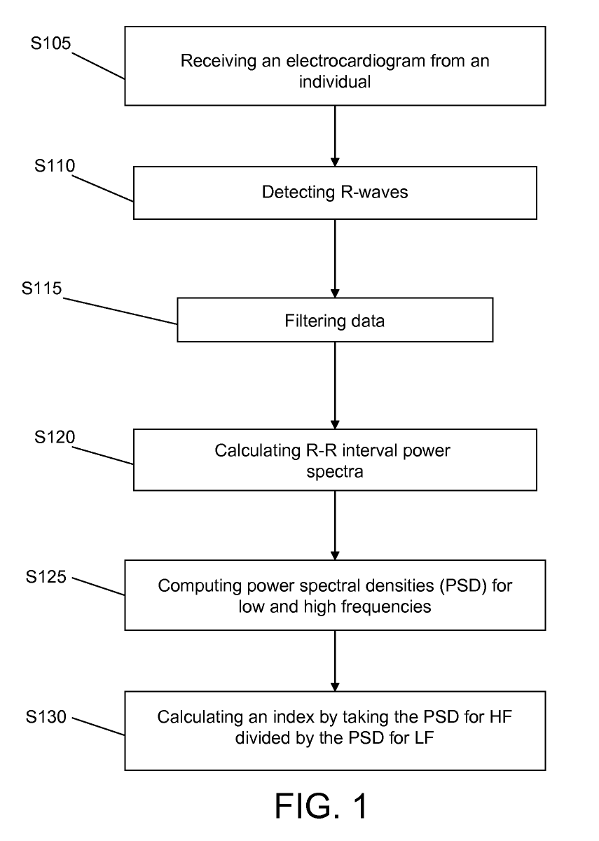

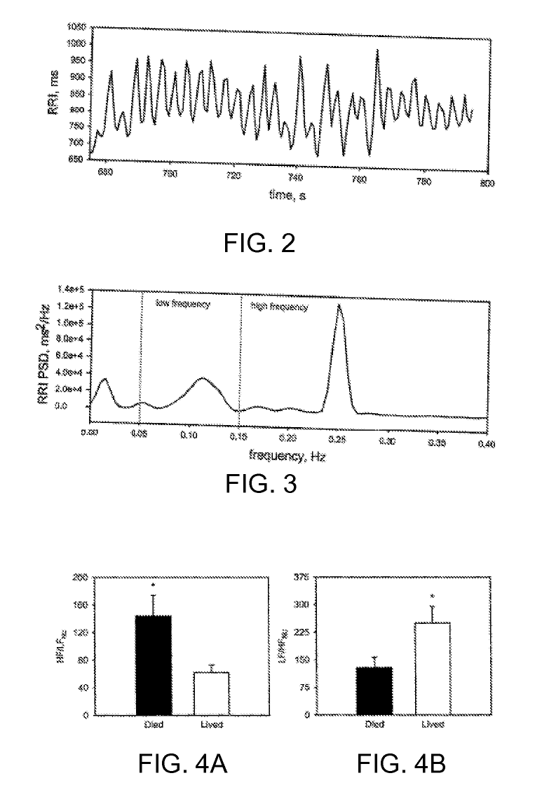

FIG. 1 illustrates an exemplary method for determining an index for a patient based on a ratio of high frequency power versus low frequency power. The method begins by receiving (or recording depending upon the implementation) an electrocardiogram (ECG) from an individual, S105. R-waves are detected within the electrocardiogram, S110. An exemplary R-R interval waveform is illustrated, for example, in FIG. 2. The electrocardiogram data is passed through a low-pass impulse response filter to obtain frequency data below a predetermined threshold such as 0.4 Hz, S115.

The filtered data is submitted to a Fast Fourier Transform to calculate R-R interval power spectra, S120. An exemplary R-R internal power spectra is illustrated in FIG. 3. The heart rate variability alternatively may be calculated in the frequency domain using other, more non-standard procedures such as autoregressive modeling, complex demodulation and fractal dimensions instead of a Fast Fourier Transform.

A power spectral density (PSD) is computed for a range of low frequencies (LF), which for this exemplary embodiment will be 0.04 Hz to 0.15 Hz, and a set of high frequencies (HF), which for this exemplary embodiment will be 0.15 Hz to 0.4 Hz, S125. Power spectral analysis expresses the variability (variance) of the signal (R-R interval) as a function of frequency. The index for the individual is calculated by taking the PSD for HF divided by the PSD for LF to obtain a number, S130, representing the relative activity level of the parasympathetic nervous system versus the activity level of the sympathetic nervous system. The index is an indication of parasympathetic predominance of heart rate control. An elevated HF/LF represents the beginnings of autonomic failure.

In at least one exemplary embodiment, the PSD for HF and the PSD for LF are both divided by the total power for the frequencies between 0.05 to 0.4 Hz (or the range of frequencies covered by LF and HF) then multiple by 100. The power attributable to the frequencies between 0.0 and 0.05 Hz is ignored as this frequency range is not indicative of sympathetic or parasympathetic control of the heart, but results from circadian rhythms and other low frequency attributes. This will normalize the PSD by the total signal variance to produce LFₙᵤ and HFₙᵤ. The ratio of HFₙᵤ/LFₙᵤ is an indication of parasympathetic predominance of heart rate control, and the ratio of LFₙᵤ/HFₙᵤ is an indication of sympathetic predominance of heart rate control. Based on prior studies (some of which are discussed later in the examples) leading to this method, patients who survive had lower parasympathetic predominance as represented by the ratio of HF/LFₙᵤ. FIG. 4A illustrates results from 15 patients who died en route to a hospital compared to 15 patients with similar injuries who survived.

In at least one exemplary embodiment, the reverse ratio of LF/HF is used. Either raw PSD or normalized PSD values can be used for LF and/or HF. This ratio provides a different version than the index discussed above. Based on prior studies leading to this method, patients who survive had higher sympathetic predominance as represented by the ratio of LF/HF. FIG. 4B illustrates results of LF/HFₙᵤ from 15 patients who died en route to a hospital compared to 15 patients with similar injuries who survived.

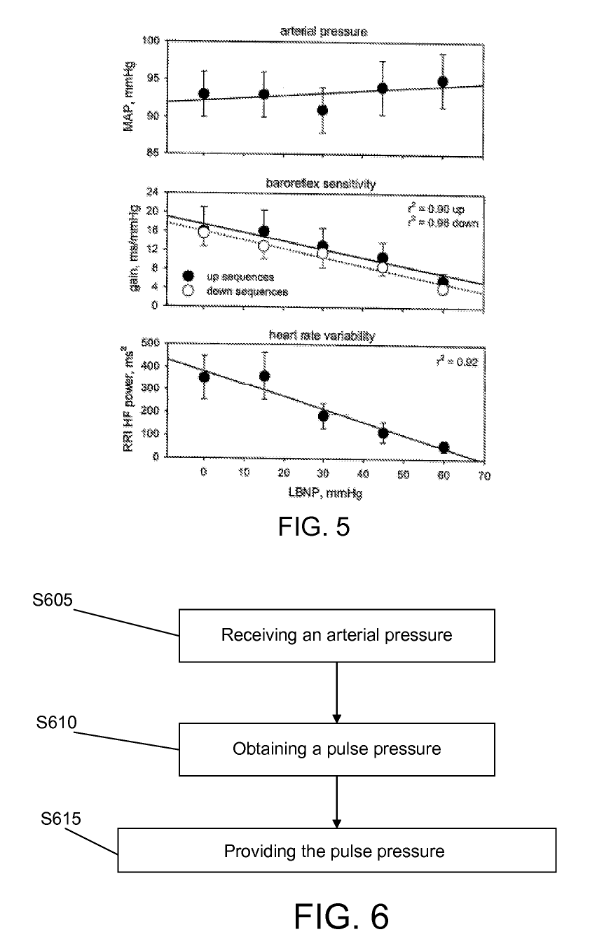

Alternatively, in place of the calculation of the index the trends associated with HF may be utilized instead. The raw or normalized HF is useable for this alternative embodiment. As discussed later, as a hemorrhage occurs and the total blood loss increases, the heart rate variability as represented by the power spectrum of the high frequencies decreases in response to the hemorrhage as illustrated, for example, in FIG. 5. As such, the trend of the power spectral density of the high frequencies is indicative that the individual is approaching cardiovascular collapse.

In at least one exemplary embodiment, the intervals between consecutive R-waves (R-R intervals) are made equidistant by spline interpolation and resampling at a frequency of 5.0 Hz. Equidistant data then is passed through the low-pass impulse response filter in step S115.

In at least one exemplary embodiment the frequency ranges are adjusted for a particular individual to account for respiratory component influences. The high frequencies band is generally thought of as the respiratory frequencies with the peak indicating the patient's respiratory frequency.

In at least one exemplary embodiment the baroreflex sensitivity is determined based on the arterial pressure and the electrocardiogram for the individual. An exemplary way to measure arterial pressure on a beat-by-beat basis is by measuring it with finger photoplethysmography. The information to be obtained from the electrocardiogram includes R-R intervals. The baroreflex sensitivity will be based on linear regression analysis on three or more progressively increasing and decreasing systolic pressures and corresponding increasing and decreasing R-R intervals. Baroreflex sensitivity is expressed as milliseconds change in R-R interval per mmHg change in systolic pressure of the individual. Baroreflex sensitivity is a cardiovascular collapse indicator over time based on its trend. If it is trending lower as illustrated, for example, in FIG. 5, then the individual is more likely to have a cardiovascular collapse.

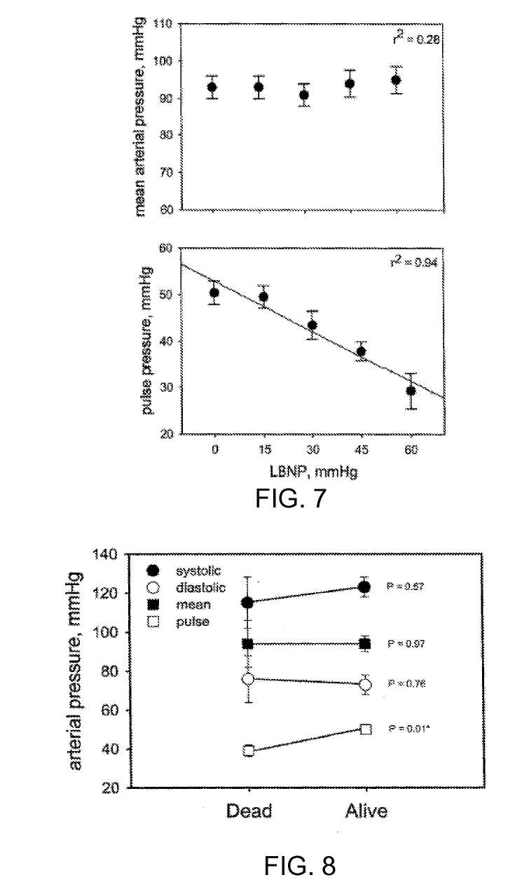

An exemplary method for determining pulse pressure is illustrated in FIG. 6. The method begins with receiving (or recording depending upon the implementation) an arterial pressure, S605. The diastolic pressure is subtracted from the systolic pressure to provide the pulse pressure, S610. The pulse pressure is then provided, S615. In at least one exemplary embodiment, this method occurs in real time. An exemplary pulse pressure over time is illustrated in FIG. 7 showing an inverse relation with the level of hemorrhage simulation. FIG. 8 illustrates that pulse pressures are significantly lower in 15 trauma patients who die compared to 15 trauma patients who live, and that the difference is statistically significant (p=0.01). While in contrast the systolic pressure (p=0.57), the diastolic pressure (p=0.76), and mean arterial pressure (p=0.97) are not significantly different.

Pulse pressure can be a surrogate for stroke volume and subsequently as a means to track loss of blood volume in trauma patients. Monitoring of pulse pressure could be an easily obtained surrogate of stroke volume, essentially an early warning measure, alerting medical personnel that casualties appearing stable, may in fact be approaching cardiovascular collapse. These noninvasive easily acquired data may be even more useful as triage tool in a mass casualty situation where effective triage decisions depend on accurate prioritization.

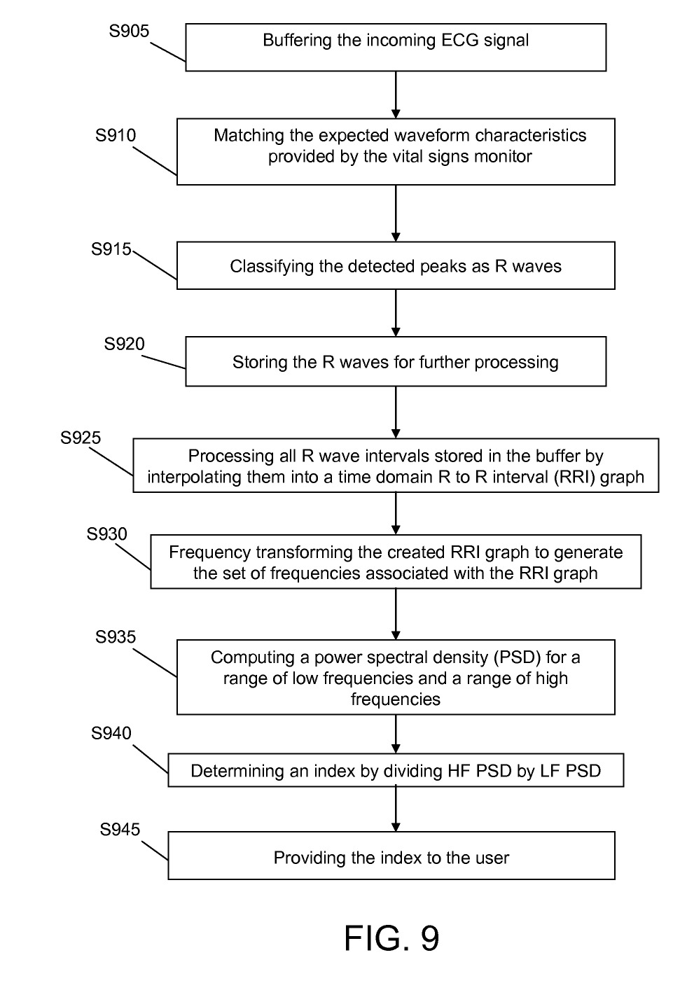

FIG. 9 illustrates another exemplary method for determining heart rate variability according to the invention. The method begins with buffering the incoming ECG signal into an internal memory buffer, S905. As the buffer is processed, matching the expected waveform characteristics provided by the vital signs monitor, S910, using, for example, a matched filter set. An exemplary device uses a 5 point kernel to amplify the waveform section peaks. Classifying the detected peaks as R waves, S915, and storing them for further processing, S920. When the buffer is full, processing all R wave intervals stored in the buffer by interpolating them into a time domain R to R interval (RRI) graph, S925.

Frequency transforming the created RRI graph to generate the set of frequencies associated with the RRI graph, S930, using, for example, a Fast Fourier Transform. In at least one exemplary embodiment, the frequency transforming step includes applying either a Hamming or Hanning digital signal filter to smooth the buffer edges before transforming the RRI graph.

Computing a power spectral density (PSD) for a range of low frequencies, LF, which in this example is 0.04 Hz to 0.15 Hz, and a set of high frequencies, HF, which in this example is 0.15 Hz to 0.4 Hz, S935. One of ordinary skill in the art will appreciate based on this disclosure that these frequency ranges can be adjusted to account for respiratory component influences of the patient.

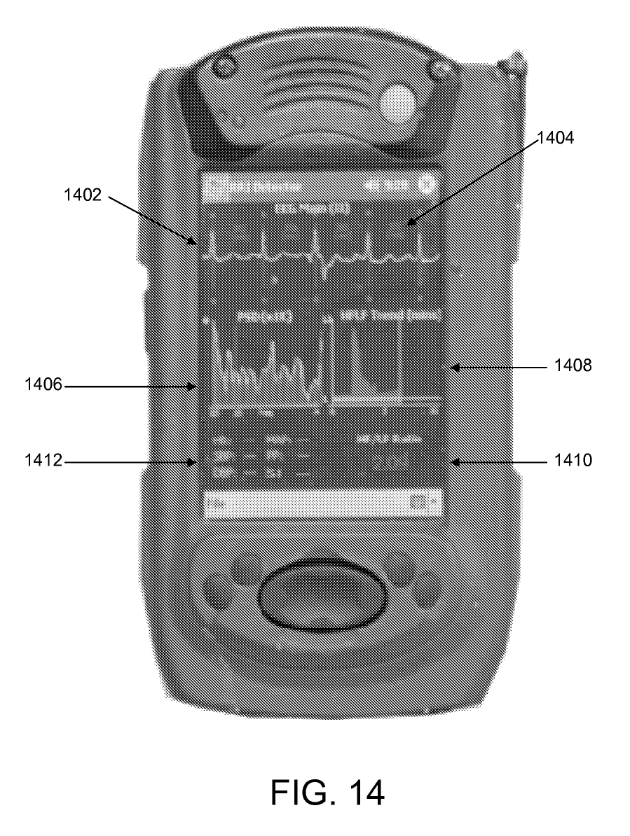

Determining an index by dividing HF PSD by LF PSD, S940. Providing the index to the user, S945. Exemplary ways for providing the index include displaying it for the user as a discrete number and graphically with prior index values allowing for visual determination of trends; displaying the information using color codes with a representation of the current index; providing audio notification; and providing text message. An exemplary color coding scheme is if the index value is less than 0.8, then displaying a green indicator; if the index value is between 0.8 and 1.2, then displaying a yellow indicator; and if the index value is greater than 1.2, then displaying a red indicator to represent possible mild, moderate, or severe changes in the heart rate variability of the individual which may be indicative of the individual's status. Alternatively, an index below 1.0 is indicative of little current risk while an index above 1.0 indicates the possibility of a cardiovascular collapse occurring. In the exemplary display illustrated in FIG. 14, the indicator is having the index value displayed in color, which in the illustrated example is red since the index value is 2.09.

In another exemplary embodiment, the method also updates a trend buffer on the display, which provides information to the user. The method also readjusts the display range based on the current maximum and minimum values stored in the trend buffer to maximize resolution on the display. Alternatively, the historical values can be stored in addition to or instead of using the trend buffer.

In another exemplary embodiment, the method also provides generated spectral decomposition to the user. Examples of this are the graphical display in area 1406 in FIG. 14 and the graphical representation shown in FIG. 3.

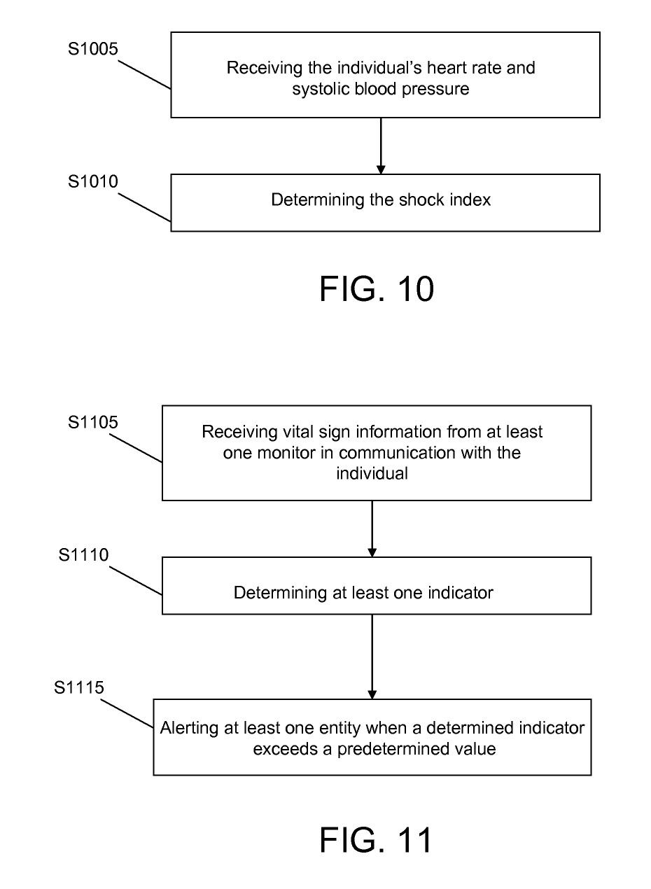

A further exemplary embodiment determines a shock index for the individual. FIG. 10 illustrates an exemplary method for determining the shock index. Receiving the individual's heart rate and systolic blood pressure, S1005. Determining the shock index by dividing the heart rate by the systolic blood pressure, S1010. Providing the shock index to the user, S1005.

FIG. 11 illustrates an exemplary method for monitoring at least one individual remotely. Receiving vital sign information from at least one monitor in communication with the individual, S1105. In communication includes having the monitor affixed, attached, implanted, coupled, abutting the individual's tissue, resident in clothing or equipment worn by the individual, and proximate to the individual.

Determining at least one indicator selected from the heart rate variability index, the baroreflex sensitivity trend, the pulse pressure value, and the shock index for the individual, S1110. Different implementations of the method may include one of the indicators or a subset of the indicators system-wide or based on the individual.

When a determined indicator exceeds a predetermined value, alerting at least one entity, S1115. In at least one exemplary embodiment, the heart rate variability index, the pulse pressure value and the shock index may have a numerical number as the predetermined value. In at least one exemplary embodiment, the slope of the trend of at least one of the heart rate variability index, the baroreflex sensitivity, the pulse pressure, and the shock index is the predetermined value and as such the trend for the indicator is tracked over time. Exemplary alerts include audio, vibration, changed display or other similar type of notification. Exemplary entities include supervisors, commanders, medical personnel, monitors, first responders, recovery personnel, and computerized monitoring and/or command system including artificial intelligence. In at least one exemplary embodiment, the method further includes prioritizing individuals based on at least one indicator for at least one of assistance, evacuation, and routing once evacuated.

An alternative embodiment where the individual has vital signs processing as part of worn equipment, for example, Warfighter Physiological Status Monitor (WPSM), the method includes receiving at least one indicator selected from the heart rate variability index, the baroreflex sensitivity trend, the pulse pressure value, and the shock index for the individual in addition to or in place of vital sign information.

In a further alternative embodiment, the method is performed by equipment worn by the individual with the notification being sent to another person or entity. For either this alternative embodiment or the method illustrated in FIG. 11, the notification can be provided via the Battlefield Medical Information System-Telemedicine (BMIST), which is a point-of-care handheld assistant for medics and other first responders.

In a further alternative embodiment, the method includes the user initiating a communication session with the vital signs monitor associated with an individual to determine the individual's current state. After the individual's current state can be determined, ending the communication session. If the user is remote from the individual, establishing the communication session wirelessly. The communication session also may be established with a wired connection be connecting the device to the vital signs monitor on the individual using, for example, a RS-232 serial protocol, an USB connection, a firewire connection, or other similar types of connections.

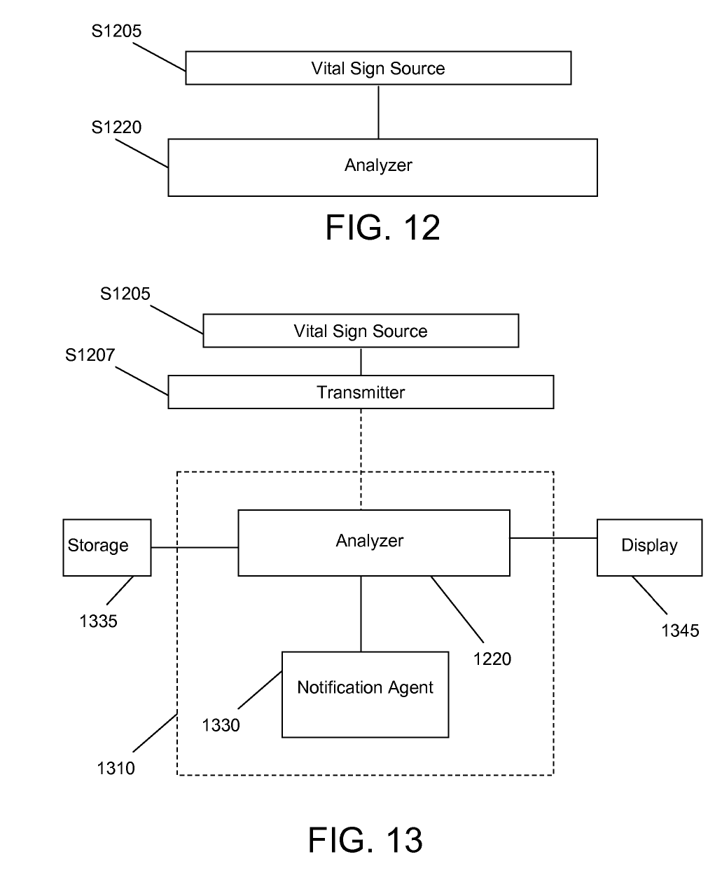

FIG. 12 illustrates a conceptual design for a system for performing the methods discussed above. The illustrated system includes a vital sign source 1205 and an analyzer 1220 in communication with the vital sign source 1205. The system although illustrated with one vital sign source 1205 may be expanded to include a plurality of vital sign sources 1205 connected to one individual and/or multiple individuals. In at least one exemplary embodiment, an individual would have multiple vital sign sources connected to monitor different vital signs for the system. The system also is designed to handle monitoring of multiple individuals.

Exemplary vital sign sources 1205 include the WPSM, a vital sign monitor (or sensor), a BMIST unit, or similar devices. The vital sign monitor will be in communication with an individual where in communication includes having the monitor affixed, attached, implanted, coupled, abutting the individual's tissue, resident in clothing or equipment worn by the individual, and proximate to the individual.

The analyzer 1220 is in communication with the vital sign source 1205 through a wired connection or wireless connection such as infrared, radio, Bluetooth, and Wi-Fi where the connection can be continual, intermittent (or on a predetermined schedule), as needed or as permitted by the circumstances. The analyzer 1220 may be a separate component not present on the individual on whom the vital sign source 1205 is present or in communication with, for example, to allow remote monitoring of the individual or monitoring during a medical event such as triage, transport, or treatment. In this implementation, the vital sign source 1205 is connected to a transmitter (and/or receiver) 1207 that allows vital sign data to be communicated to the analyzer 1220 as illustrated in FIG. 13. Alternatively, the analyzer 1220 may be located on (or proximate to) the individual whom the vital sign source 1205 is in communication, and in this implementation an exemplary system for the analyzer 1220 to be configured as part of is the WPSM or other individual centric monitoring system that is capable of communicating with a remote user. If the analyzer 1220 is located on the individual, then in at least one exemplary embodiment the analyzer 1220 is connected to a transmitter (and/or receiver) 1207.

The analyzer 1220 processes received vital sign data from the vital sign source 1205. Depending upon the implementation, the set of vital sign data includes heart rate data such as ECG and/or arterial pressures to be able to determine the heart rate variability index, baroreflex sensitivity, pulse pressure, and/or shock index.

The analyzer 1220 as illustrated in, for example, in FIG. 14, can be implemented on a variety of computing devices including computers and PDAs as software. The software includes the ability to process the received signals to provide as an output the desired indicators relating to cardiovascular collapse. As illustrated in FIG. 13, the software 1310 when used to implement the method illustrated in FIG. 11, includes notification/alarm agent(s) 1330 to provide notification to the user with an audio notification, a mechanical notification such as vibration, a visual notification including activation of a light(s) or via the display, signal to another entity or device, or any combination of these if predetermined conditions occur or predetermined thresholds are exceed by a vital sign or an indicator. The analyzer 1220 in at least one exemplary embodiment is connected to storage 1335 including a buffer, RAM and disk storage for storing data associated with its operation.

The exemplary graphical user interface shown in FIG. 14 includes areas for displaying the ECG waveform 1402, the RRI time spread in milliseconds 1404, ECG frequency decomposition of the PSD with a low frequency and high frequency breakout 1406, heart variability index trend graphical representation 1408, the current heart rate variability index 1410, and other vital signs 1412. Exemplary vital signs that might also be displayed in area 1412 include heart rate, systolic blood pressure, diastolic blood pressure, and mean arterial pressure. The other vital signs section 1412 may also display determined numbers like the pulse pressure and stroke index along with fields providing observational information like pulse character and mental status. The display also could be arranged to display baroreflex sensitivity trend information. Other possible vital signs that may be displayed if available include blood oxygen level percentage (SpO₂) and end-tidal carbon dioxide (EtCO₂). Based on this disclosure, it will be realized that a variety of information can be displayed for the user.

The analyzer 1220 can work in conjunction with or be an additional component of the BMIST system that is PDA based to record the medical information being collected for later review and/or use.

Using a wireless connectivity subsystem, the system provides the user the capability of reading an individual's vital signs from a monitor attached to the individual for remote monitoring.

The analyzer 1220 in at least one exemplary embodiment initiates a connection to the vital sign source 1205 when the user is ready to start a monitoring session to conserve power in the devices and/or reduce the bandwidth need. The connection is active until broken by the user or due to loss of signal from the vital sign source 1205.

The invention can take the form of an entirely hardware embodiment, an entirely software embodiment or an embodiment containing both hardware and software elements. In at least one exemplary embodiment, the invention is implemented in software, which includes but is not limited to firmware, resident software, microcode, etc.

Furthermore, the invention can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium such as carrier signal. Examples of a computer-readable medium include a semiconductor or solid state memory, magnetic tape, a removable computer diskette, a random access memory (RAM), a read-only memory (ROM), a rigid magnetic disk, and an optical disk. Current examples of optical disks include compact disk—read only memory (CD-ROM), compact disk—read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Network adapters may also be coupled to the system to enable the data processing system to become coupled to other data processing systems or remote printers or storage devices through intervening private or public networks. Modems, cable modem and Ethernet cards are just a few of the currently available types of network adapters.

Computer program code for carrying out operations of the present invention may be written in a variety of computer programming languages. The program code may be executed entirely on at least one computing device, as a stand-alone software package, or it may be executed partly on one computing device and partly on a remote computer. In the latter scenario, the remote computer may be connected directly to the one computing device via a LAN or a WAN (for example, Intranet), or the connection may be made indirectly through an external computer (for example, through the Internet, a secure network, a sneaker net, or some combination of these).

It will be understood that each block of the flowchart illustrations and block diagrams and combinations of those blocks can be implemented by computer program instructions and/or means. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions specified in the flowcharts or block diagrams.

The exemplary and alternative embodiments described above may be combined in a variety of ways with each other. Furthermore, the steps and number of the various steps illustrated in the figures may be adjusted from that shown.

It should be noted that the present invention may, however, be embodied in many different forms and should not be construed as limited to the embodiments set forth herein; rather, the embodiments set forth herein are provided so that the disclosure will be thorough and complete, and will fully convey the scope of the invention to those skilled in the art. The accompanying drawings illustrate exemplary embodiments of the invention.

Although the present invention has been described in terms of particular preferred and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the preferred and alternative embodiments described above can be configured without departing from the scope and spirit of the invention. Therefore, it is to be understood that, within the scope of the appended claims, the invention may be practiced other than as specifically described herein.

A variety of studies led to the invention discussed above. Examples of these studies are discussed below that provide a scientific basis for the invention.

### EXAMPLE 1

FIGS. 5 and 7 illustrate how heart rate variability as represented by HF, baroreflex sensitivity, and pulse pressure are direct inverse functions to increasing in the magnitude of suction applied to the lower body (LBNP) while mean arterial pressure remains constant over these same pressures. LBNP is a technique to simulate hemorrhage, as it “pulls” blood from the thorax to the dependent regions of the pelvis and legs. This simulated hemorrhage model supports the contention that power spectral analysis, baroreflex analysis and pulse pressure identification will provide a first responder with information to track hemorrhage severity—such information is currently unavailable with existing technology.

### EXAMPLE 2

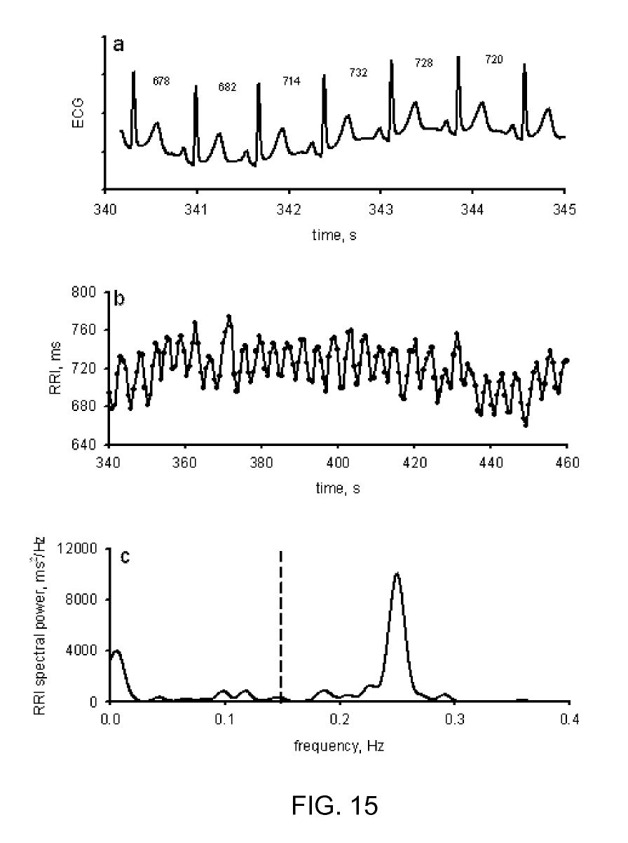

The purpose of this study was to test the hypothesis that an elevated prehospital HF/LF ratio is associated with the need for a life saving intervention (LSI). An analysis of prehospital trauma patient records collected during helicopter transport to a Level 1 Trauma center was conducted. R-waves from two minute segments of ECG waveforms were detected by computer and converted to the frequency domain with a fast Fourier transform. Analysis of variance was performed on three sets of patients, including: 1) survivors with life saving interventions (LSI) (n=36) vs. no LSI (n=36); 2) survivors (n=13) versus non-survivors (n=13) matched by injury severity score (mean ISS=23); and 3) survivors (n=37) versus non-survivors (n=42) irrespective of injury type or treatment. Fourier analysis of pre-hospital ECG collected en route to a level one trauma center was done and an exemplary set of ECG data is illustrated in FIG. 15, which shows the ECG data versus time, R-R interval data versus time, and R-R interval spectral power versus frequency, which then is used to determine the index of HF/LF.

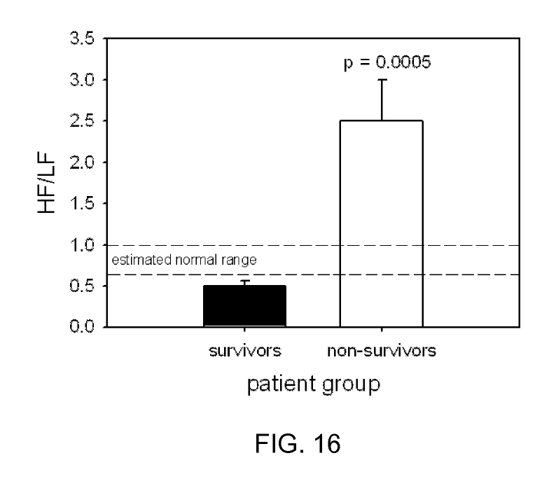

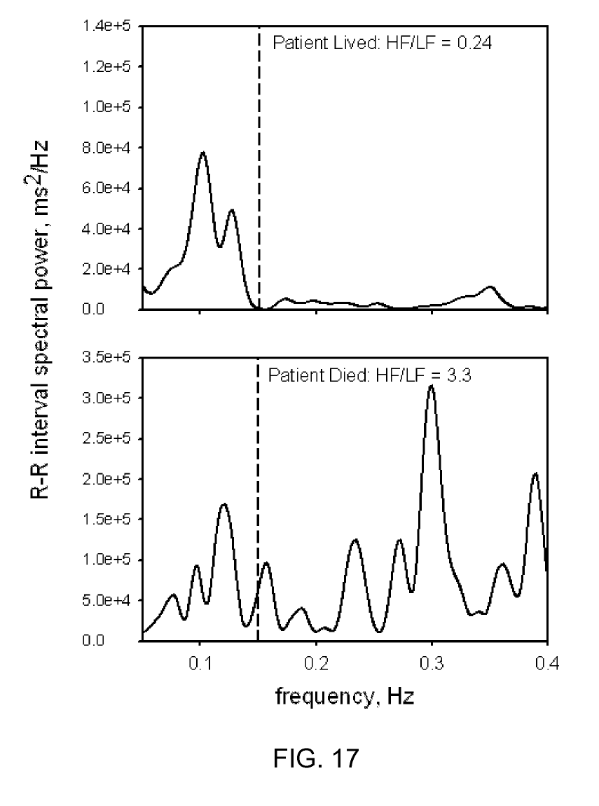

The HF/LF tended to be normal (˜1.0) in patients who did not receive an LSI and lived versus those survivors who received an LSI (1.1±2.2 vs. 0.53±0.4; p=0.15). The HF/LF ratio tended to be higher in non-survivors compared to survivors matched for injury severity score (1.9±0.7 vs. 0.9±0.2; p=0.14), and was increased significantly in non-survivors versus survivors when the entire patient cohort was considered as a whole (2.5±0.5 vs. 0.5±0.07; p=0.0005). The HF/LF ratio was elevated 19 hours (median) before death, when systolic pressure was not different between survivors and non-survivors (120±5.1 vs. 121±6.1; p=0.89). HF/LF between groups: 1) survivors requiring LSI (0.53±0.07) versus no LSI (1.1±2.2) p=0.15; 2) survivors (0.9±0.2) versus non-survivors (1.9±0.7) matched for injury severity p=0.14; and 3) survivors (0.5±0.07) versus non-survivors (2.5±0.5) irrespective of injury p=0.0001. FIG. 16 illustrates a graphical representation of all of the survivors versus all of the non-survivors with an estimated normal range superimposed. FIG. 16 reflects a p=0.0005. Systolic pressures for group 3 survivors was 120±5.1 and non-survivors was 121±6.1 (p=0.89). FIG. 17 illustrates two examples of R-R interval spectral power, the top graph is of a patient who survived and had an index of 0.24 and the bottom graph is of a patient who died and had an index of 3.3.

An elevated HF/LF ratio derived from frequency domain analysis of heart rate variability represents inappropriate parasympathetic predominance in trauma patients, and may be useful as a diagnostic tool. Parasympathetic neural activity is elevated in non-survivors versus survivors irrespective of injury type or mechanism. Heightened parasympathetic activity occurs at a point in time during helicopter transport when arterial pressures are similar. Heart rate variability analysis represents a new vital sign that may provide advanced recognition of injury severity.

### EXAMPLE 3

The experiment used a lower body negative pressure machine (LBNP) to simulate hemorrhage in humans and had two parts with uncontrolled breathing and controlled breathing. Absolute equivalence between the magnitude of negative pressure applied and the magnitude of actual blood loss cannot at this time be determined, but review of both human and animal data reveal ranges of effective blood loss (or fluid displacement) caused by LBNP. On the basis of the magnitude of central hypovolemia induced, it has previously been proposed that ten to 20 mmHg negative pressure is equivalent to blood loss ranging from 400 to 550 ml; 20 to 40 mmHg negative pressure is equivalent to blood loss ranging from 550 to 1,000 ml; and greater than 40 mmHg negative pressure is equivalent to blood loss approximating 1,000 ml or more.

For part one of the experiment, subjects underwent an LBNP protocol consisting of a 12 minute baseline period followed by exposure to −15, −30, −45, and −60 mmHg decompression for 12 minutes each, followed by return to baseline (0 mmHg) for 12 minutes. For three minutes during each stage, subjects controlled their breathing rate at a strict 15 breaths per minute (0.25 Hz) for the purpose of assessing heart rate variability. Breathing at 15 breaths per minute may be faster than subjects' normal un-paced breathing rate, but the purpose was to insure that oscillations of R-R intervals occurring at the respiratory frequency were not confounded inappropriately by harmonics of low frequency rhythms occurring around 0.1 Hz. During this experiment, the first two minutes of each stage were used for experimental retinal scans (data not presented), and subjects responded verbally to instructions from the investigators. For this reason, there is no data to compare heart rate variability during uncontrolled spontaneous versus controlled frequency breathing during LBNP.

For part two of the experiment, subjects were supine for a five-minute stabilization period, and data then were recorded with subjects breathing spontaneously, at an uncontrolled rate for five minutes. Following this, subjects breathed in time to a metronome set at a pace of 15 breaths per minute (0.25 Hz) for an additional five minutes. These data were used to assess the influence of controlled frequency breathing on heart rate variability and spontaneous baroreflex sequences and cardiac baroreflex sensitivity (BRS).

Heart rate variability was assessed in the frequency domain from R-R interval spectral power. R-R intervals were made equidistant by spline interpolating and resampling at 5 Hz. Data then were passed through a low-pass impulse response filter with a cutoff frequency of 0.5 Hz. Three minute data sets (experiment 1) and five minute data sets (experiment 2) were fast Fourier transformed with a Hanning window to obtain power spectrums. Heart rate variability was quantified as the total integrated area within the high-frequency (0.15-0.4 Hz) band.

Automated computer analysis was used to search the entire data records for potential baroreflex sequences. A potential valid sequence was defined as three or more progressively increasing or decreasing systolic pressures with at least one mmHg change per beat and associated R-R intervals with at least four milliseconds change per beat. Sequences of increasing systolic pressures and R-R intervals were classified as ‘up sequences’ and decreasing systolic pressures and R-R intervals were classified as ‘down sequences’. Cardiac baroreflex sensitivity (gain) was estimated with linear regression analysis. Only sequences with correlations of greater than or equal to 0.8 were considered to be valid sequences and included in the analysis.

All data were analyzed with commercial statistical software (SAS Institute, Cary, N.C.). For part one of the experiment, regression coefficients between LBNP and R-R interval high frequency power, spontaneous baroreflex sequences, and mean arterial pressure were calculated. In addition, analysis of variance for repeated measures was used to compare heart rate variability at baseline to heart rate variability during the highest LBNP level (−60 mmHg). For part two of the experiment, differences between the means of each dependent variable were tested with a two way analysis of variance with repeated measures on both condition (uncontrolled breathing vs. controlled breathing) and time (one minute periods). Significance was set at p≦0.05. Data are presented as means ± standard error (SE) unless specified otherwise.

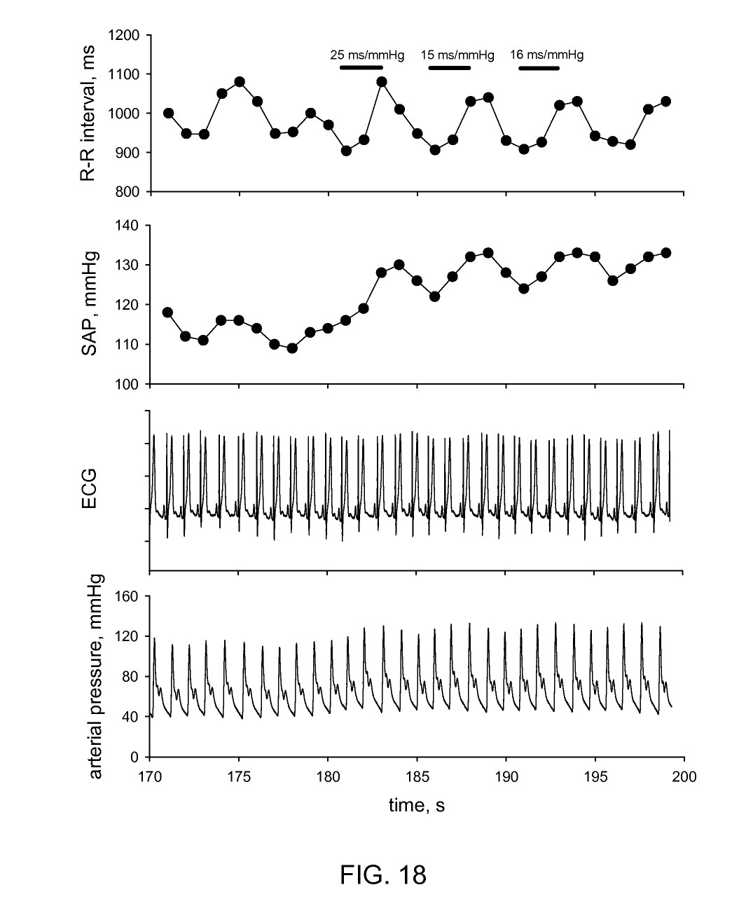

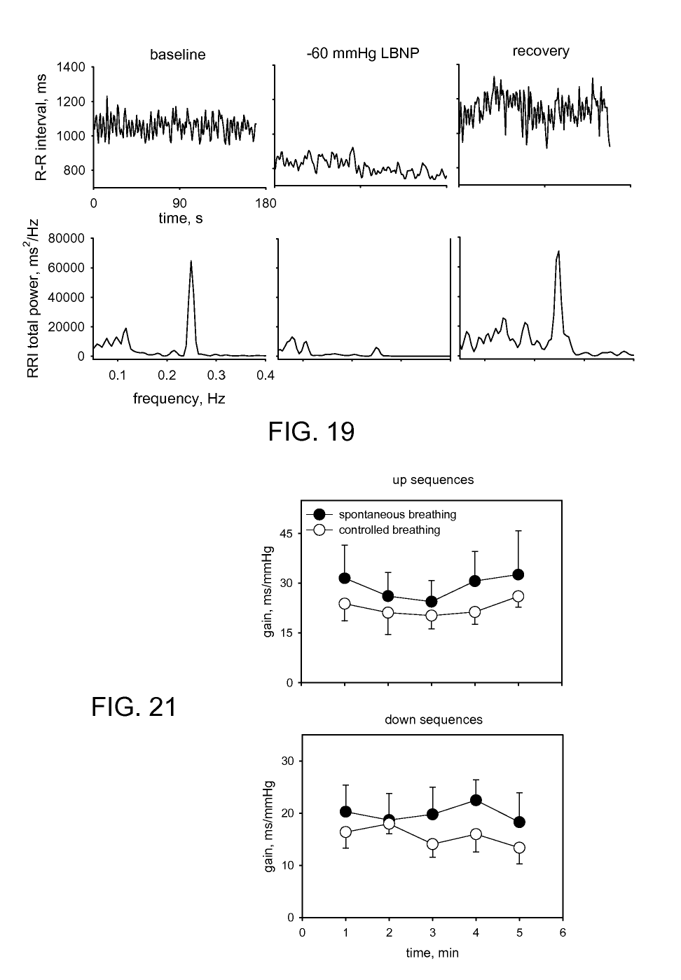

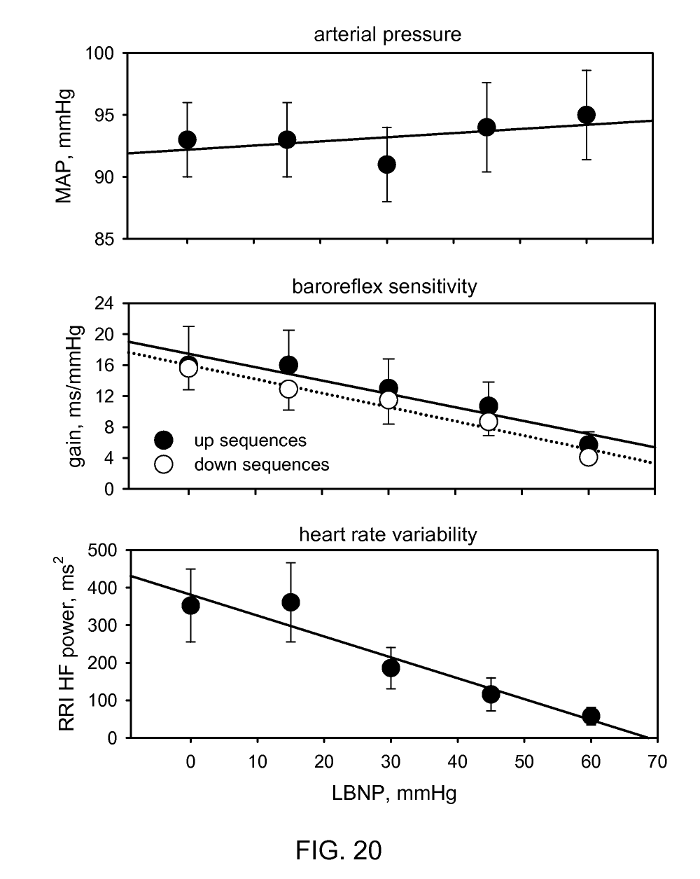

FIG. 18 shows original tracings of arterial pressures and ECG. Systolic arterial pressures (SAP) and R-R intervals are shown in the upper two panels with valid up sequences marked in the R-R interval panel. FIG. 19 shows R-R intervals and associated frequency domain representations for one representative subject during baseline (0 mmHg), −60 mmHg decompression, and return to 0 mmHg. The data shown in FIG. 19 represent an example of the magnitude of reduction in R-R interval spectral power at −60 compared to 0 mmHg chamber decompression. As a group (n=10), the average magnitude of reduction in heart rate variability from 0 mmHg to −60 mmHg was similar to the example shown in FIG. 19, and was statistically significant at the p=0.0001 level. For all subjects, LBNP caused progressive decreases in both heart rate variability and BRS of both up and down baroreflex sequences as shown in FIG. 20. Heart rate variability and BRS were correlated inversely to LBNP level (r²=0.92 for LBNP and heart rate variability; r²=0.90 for LBNP and baroreflex up sequences; r²=0.96 for LBNP and baroreflex down sequences). Mean arterial pressure did not change predictably with progressive LBNP (r²=0.26 for LBNP and mean arterial pressure).

Breathing frequency was not significantly different between uncontrolled and controlled frequency breathing. Average respiratory rate was 15.5±0.9 breaths per minute during spontaneous breathing, and exactly 15±0.0 breaths per minute (by design) during controlled breathing (P=0.8).

Controlled breathing did not affect estimates of vagal-cardiac control. R-R intervals were 953±26 ms during uncontrolled breathing and 942±29 ms during controlled breathing (p=0.2). R-R interval standard deviations were 69±8 ms during uncontrolled breathing and 65±7 ms during controlled breathing (p=0.1). R-R interval high frequency power was 1837±573 ms² during uncontrolled breathing and 1410±339 ms² during controlled breathing (p=0.3).

Controlled breathing did not affect the number of up or down baroreflex sequences or BRS. An average of 13 potential up sequences and 11 potential down sequences were detected during the five minute periods of both uncontrolled and controlled breathing. The percentage of these sequences that were determined to be valid up sequences (those with r≧0.8) were not affected by controlled breathing (6.4±0.8% uncontrolled vs. 7.8±1.3% controlled; p=0.4), nor were the percentages of valid down sequences (6.8±0.9% uncontrolled vs. 6.6±0.8% controlled; p=0.9). Cardiac baroreflex sensitivity calculated for up sequences (29±4.1 ms/mmHg uncontrolled vs. 21±2.1 ms/mmHg controlled; p=0.9) and down sequences (21±2.2 ms/mmHg uncontrolled vs. 17 ms/mmHg controlled; p=0.1) statistically were indistinguishable between the two conditions.

No condition by time interaction effects was found. FIG. 21 shows characteristics of baroreflex gains across time, and Tables 1 (characteristics of up sequences during uncontrolled, spontaneous and controlled frequency breathing) and 2 (characteristics of down sequences during uncontrolled, spontaneous and controlled frequency breathing) show the number of up and down sequences identified, the percentage of valid sequences, and the mean BRS for each minute during each condition.

TABLE 1

| | | # sequences | | Mean, |
|---|---|---|---|---|
| | Time, min | (range) | % valid | ms/mmHg (n) |
| UB | 1 | 3 (0-7) | 5.9 ± 1.9 | 31.5 ± 10.0 |
| | | | | (11) |
| | 2 | 2 (0-5) | 5.7 ± 1.7 | 26.2 ± 7.2 |
| | | | | (11) |
| | 3 | 2 (0-5) | 7.3 ± 1.9 | 24.5 ± 6.4 |
| | | | | (11) |
| | 4 | 3 (0-4) | 7.1 ± 2.0 | 30.6 ± 9.0 |
| | | | | (13) |
| | 5 | 3 (0-5) | 6.3 ± 1.9 | 32.6 ± 13.2 |
| | | | | (11) |
| CB | 1 | 2 (0-5) | 8.1 ± 1.6 | 23.8 ± 5.2 |
| | | | | (15) |
| | 2 | 3 (0-4) | 5.4 ± 1.5 | 21.8 ± 6.6 |
| | | | | (11) |
| | 3 | 3 (0-4) | 7.2 ± 2.4 | 20.2 ± 4.0 |
| | | | | (10) |
| | 4 | 3 (0-5) | 8.2 ± 3.0 | 21.3 ± 3.7 |
| | | | | (10) |
| | 5 | 2 (0-3) | 10.3 ± 4.5 | 25.4 ± 3.2 |
| | | | | (11) |

UB, controlled spontaneous breathing; CB controlled frequency breathing at 0.25 Hz; # sequences, number of valid sequences identified per subject with ranges shown in parentheses; % valid, percentage ± standard error of all potential sequences (n=20 subjets per time period) that met the criterion for a valid up sequence; Mean, arithmetic average ± standard error of all valid up sequences with the number of observations contribuing to the mean (n) shown in parentheses.

TABLE 2

| | | # sequences | | Mean, |
|---|---|---|---|---|
| Condition | Time, min | (range) | % valid | ms/mmHg (n) |
| UB | 1 | 2 (0-4) | 8.4 ± 2.1 | 20.3 ± 5.1 |
| | | | | (14) |
| | 2 | 2 (0-3) | 6.7 ± 2.1 | 18.7 ± 5.1 |
| | | | | (10) |
| | 3 | 2 (0-3) | 7.0 ± 2.4 | 19.8 ± 5.2 |
| | | | | (10) |
| | 4 | 3 (0-4) | 6.8 ± 9.6 | 22.5 ± 3.9 |
| | | | | (11) |
| | 5 | 2 (0-3) | 5.4 ± 1.8 | 18.3 ± 5.6 |
| | | | | (9) |
| CB | 1 | 2 (0-4) | 7.1 ± 1.6 | 16.4 ± 3.1 |
| | | | | (15) |
| | 2 | 2 (0-4) | 6.6 ± 1.7 | 18.5 ± 1.9 |
| | | | | (11) |
| | 3 | 3 (0-4) | 7.1 ± 2.3 | 14.2 ± 2.5 |
| | | | | (11) |
| | 4 | 2 (0-5) | 6.2 ± 1.8 | 16.2 ± 3.4 |
| | | | | (10) |
| | 5 | 2 (0-3) | 6.2 ± 1.4 | 13.4 ± 3.1 |
| | | | | (12) |

UB, uncontrolled, spontaneous breathing; CB controlled frequency breathing at 0.25 Hz; # sequences, number of valid sequences identified per subject with ranges shown in parentheses; % valid, percentage ± standard error of all potential sequences (n=20 subjects per time period) that met the criterion for a valid down sequence; Mean, arithmetic average ± standard error of all valid down sequences with the number of observations contributing to that mean (n) shown in parentheses.

The results demonstrate that heart rate variability and BRS change as direct inverse functions of LBNP magnitude while mean arterial pressures remain constant. In addition, heart rate variability and BRS are not affected importantly by controlled breathing. The conclusion is that heart rate variability and baroreflex sequence analyses accurately represent autonomic changes occurring during progressive central hypovolemia and may have greater predictive power compared to measures of arterial pressure for early identification of progression to hemodynamic instability. Accurate application of heart rate variability and baroreflex analyses does not depend on maintenance of an unchanging breathing rate, and therefore show promise as tools to assist in the assessment of hemodynamic status in bleeding patients.

The greatest utility of using LBNP as a model to replicate hemodynamic effects of hemorrhage is revealed by comparing compensatory responses among the two conditions. Both hemorrhage and LBNP induce central hypovolemia in proportion to blood loss or negative pressure applied, and the resulting compensations include sympathoexcitation to increase peripheral vascular resistance and heart rate to counteract reductions of stroke volume and defend arterial pressure. Autonomic sympathetic activation is fundamental to maintenance of hemodynamic stability under both conditions, and both hemorrhagic shock during actual hemorrhage and hemodynamic instability with high-level LBNP occur consequent to abrupt hypotension mediated by sympathetic neural withdrawal.

FIG. 20 shows that both heart rate variability and BRS change early, and in direct inverse proportion to the magnitude of LBNP (i.e., central hypovolemia). Mean arterial pressure is effectively maintained constant, and is therefore of little use in an early prediction algorithm. The results, shown in FIG. 20, suggest that changes in autonomic vagal activity could assist in the early assessment of hemorrhage severity.

Heart rate reflects average ongoing sympathetic neural traffic with a time delay of about 10 seconds due to intrinsic delays in effector responses to norepinephrine. In contrast, acetylcholine kinetics (including quick degradation by acetylcholinesterase) allow for autonomic vagal activity to modulate cardiac rate on a beat-by-beat basis in response to prevailing hemodynamic changes. Heart rate variability, as expressed in FIG. 20 as the integrated area under the high frequency R-R interval power spectrum, reflects primarily vagal-cardiac activity. Due to quick activation and inhibition of acetylcholine in response to changes in arterial distention, up baroreflex sequences reflect vagal activation, and down baroreflex sequences reflect vagal inhibition. Both up and down baroreflex sequences decrease predictably as functions of LBNP magnitude (FIG. 20), suggesting that BRS conceivably could track progression to hemodynamic instability in bleeding patients.

The finding that loss of central blood volume is associated with an acute attenuation of BRS is not without precedent. In a previous study, exposure to 50 mmHg of LBNP caused a 30% reduction in BRS that was reversed when central blood volume was restored with the use of G-suits inflated to 50 mmHg. Similarly, BRS measured during rest and exercise has been reduced with application of LBNP (reduced central blood volume) and increased by application of lower body positive pressure (increased central blood volume). In contrast to the present investigation, BRS was measured in these previous studies by applying pulse-synchronous neck pressure stimuli that allowed assessment of the isolated carotid-cardiac baroreflex response over most of the reflex operational range. Findings of the present study extend those of previous experiments by demonstrating that spontaneous baroreflex sequences that reflect an integrated response of numerous baroreflexes may provide a simple, noninvasive early marker of acute alterations in central blood volume.

The attenuation of BRS may provide an important early marker for progression to hemodynamic instability. In the presence of an average 15% reduction in blood volume in subjects confined to bed rest, the largest reductions in cardiovascular insufficiency (hypotension and vasovagal syncope) were correlated with the greatest magnitude of reduction in vagal baroreflex gain. In a similar fashion, low BRS represented one of the primary contributing factors to the prediction of early cardiovascular collapse. Thus, the findings of a linear relationship between reduced BRS and central hypovolemia may be the first to suggest that spontaneous baroreflex sequences represents an early and continuous predictor of progression to hemodynamic instability.

Although the potential of using changes in spontaneous baroreflex sequences as a marker of blood loss is attractive, the limitation of the central hypovolemia model of LBNP fails to include the loss of blood volume associated with hemorrhage. With this model, there is no hole in a vessel. Of concern is the observation that an average reduction of approximately 500 ml of blood volume had no effect on carotid-cardiac BRS, suggesting that BRS may be influenced by fluid redistribution within the vascular space rather than by actual volume reduction. An attenuated BRS during LBNP is consistent with evidence that supports the existence of a muscle chemoreflex that would act to decrease systemic arterial pressure when circulation to the legs is improved. Decreased heart rate response to baroreceptor stimulation (i.e., attenuated cardiac baroreflex gain) could represent one mechanism by which the muscle chemoreflex reduces arterial pressure. Results from animal hemorrhage models coupled with the recent observations in humans, provides evidence that the reduction in BRS observed in the LBNP model may indeed represent a phenomenon of central hypovolemia rather than a chemoreflex response.

Breathing frequency was not different during spontaneous and controlled breathing in the present study, but frequencies ranged from 10 to 20 breaths per minute when subjects were allowed to breathe spontaneously. That R-R interval spectral power, R-R intervals, and R-R interval standard deviations were indistinguishable statistically between the two conditions suggests that such noninvasive measures of vagal-cardiac control are appropriate even when breathing frequencies vary widely about a mean. The data show that breathing at a fixed rate has no effect on the occurrence of baroreflex sequences, on the percentage of valid sequences, or on the sensitivity of the baroreflex response. This is true for both up and down sequences as shown in Tables 1 and 2 and FIG. 21.

Heart rate variability was assessed with standard Fourier analysis, and baroreflex sensitivity with sequence analysis primarily because these techniques have been appropriately vetted in the literature and extensive past experience analyzing and interpreting results of such analyses. The results support the use of heart rate variability and baroreflex sequence analysis as potential markers of hemorrhage severity based on a hemorrhage model incorporating LBNP. The results show that heart rate variability and baroreflex sequence analyses are not confounded when responses during spontaneous breathing (with frequencies ranging from 10 to 20 breaths per minute) are compared to responses during controlled breathing at a set rate of 15 breaths per minute. The analysis of heart rate variability and baroreflex sequences during hemorrhage can serve as an important adjunct to monitoring of pulse and blood pressure and be a reliable technique to track early autonomic changes occurring in bleeding patients during progression to hemodynamic instability.

### EXAMPLE 4

For this study, the mean arterial blood pressure (MAP), pulse pressure, stroke volume, and muscle sympathetic nerve activity (MSNA) in human subjects during progressive lower body negative pressure (LBNP) were measured to test the hypothesis that a reduction in pulse pressure tracks the reduction of stroke volume and change in MSNA during graded central hypovolemia in humans. The method was that after a 12 minute baseline data collection period, 13 men were exposed to LBNP at −15 mmHg for 12 minutes followed by continuous stepwise increments to −30, −45, and −60 mmHg for 12 minutes each. For each 12 minute step, the first 2 minutes were used to allow the subject to reach a steady-state status without data collection. Each subject breathed in time to a metronome set at a pace of 15 breaths per minute, and did not deviate from this controlled breathing frequency during the period of data collection. The stroke volume was measured using thoracic electrical bioimpedence. Muscle sympathetic nerve activity (MSNA) was measured directly with a Nerve Traffic analyzer according to the procedures described in Cooke, W. H., “Topical anesthetic before microneurography decreases pain without affecting sympathetic traffic,” Autonomic Neuroscience Basic Clin., 2000, 86:120-126.

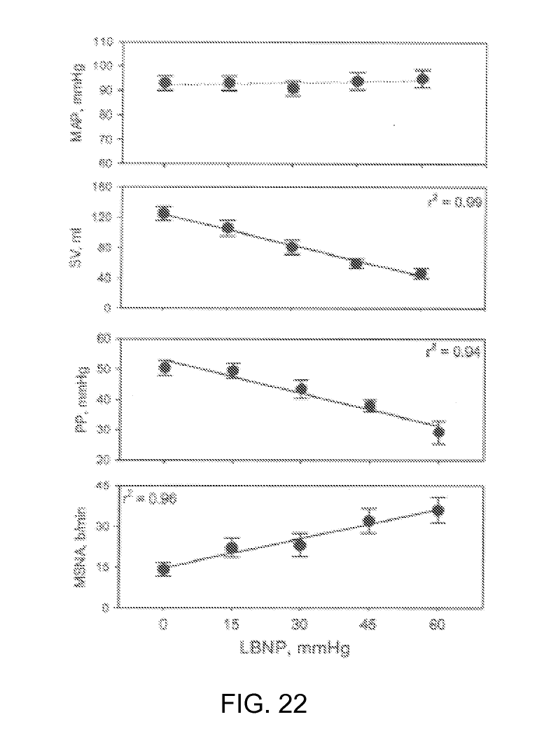

Comparing baseline to −60 mmHg chamber decompression, systolic blood pressure (SBP) decreased from 129±3.0 to 111±6.1 mmHg (p=0.005) and diastolic pressure was unchanged from 78±3.0 versus 81±4.0 mm HG (p=0.55). Pulse pressure decreased from 50±2.5 to 29±4.0 mmHg (p=0.0001). LBNP caused linear reductions in pulse pressure and stroke volume from 125±9.2 to 47±6.4 (r²=0.99), and increase in MSNA from 14±3.5 to 36±4.6 bursts/minute⁻¹ (r²=0.96) without a significant change in MAP (r²=0.28). Pulse pressure was inversely correlated with MSNA (r²=0.88) and positively correlated with stroke volume (r²=0.91). FIG. 22 shows the relationship between progressive increases in LBNP and mean (±SE) MAP, stroke volume, pulse pressure, and MSNA. As a group, LBNP caused linear reductions in pulse pressure (r²=0.94) and stroke volume (r²=0.99) and increases in MSNA (r²=0.96) without a significant change in MAP (r²=0.28). Pulse pressure is positively correlated with stroke volume (r²=0.91). The relationship between stroke volume and pulse pressure appeared to tighten using a third-order rather than simple linear regression (r²=0.98).

Pulse pressure decreases early and in a linear fashion with the magnitude of central hypovolemia with no change in mean arterial pressure. Decreased pulse pressure during central blood volume reduction is correlated significantly with reduced stroke volume and increased sympathetic nerve activity. These results suggest that pulse pressure is an earlier predictor of outcome from blood loss than systolic, diastolic, or mean arterial pressures. As a result, pulse pressure can assist the first responder in on-site triage, remote triage, and evacuation priority. The conclusion is that reduced pulse pressure resulting from progressive central hypovolemia is a marker of reductions in stroke volume and elevations in sympathetic nerve activity. Therefore, when systolic blood pressure is greater than 90 mmHg, pulse pressure may allow for early, noninvasive identification of volume loss because of hemorrhage and more accurate and timely triage.

### VI. INDUSTRIAL APPLICABILITY

This invention could be used for triage and monitoring of trauma patients to better allocate resources and improve the chances of saving lives based on earlier medical interventions despite a patient appearing to be fine. The invention in several embodiments provides for remote monitoring of individuals to more quickly dispatch medical assistance even when the individual is unable to request help themselves potentially due to injury.

We claim:

1. A method comprising:
receiving an electrocardiogram from at least one remote individual,
detecting R-waves within the electrocardiogram,
calculating R-R interval power spectra for the electrocardiogram,
calculating power spectral densities for a range of low frequencies and a range of high frequencies for the electrocardiogram,
calculating an index based on the power spectral density of the range of high frequencies divided by the power spectral density of the range of low frequencies, and
outputting the index.

2. The method according to claim 1, further comprising determining whether medical assistance is required for the individual based on the index.

3. The method according to claim 2, further comprising prioritizing medical assistance for the individual based on the index as compared to indexes for other individuals awaiting medical assistance.

4. The method according to claim 1, wherein calculating an index includes setting the index equal to the power spectral density of the range of high frequencies divided by the power spectral density of the range of low frequencies.

5. The method according to claim 1, wherein calculating an index includes normalizing the power spectral density of the range of low frequencies, and
setting the index equal to the power spectral density of the range of high frequencies divided by the normalized power spectral density of the range of low frequencies.

6. The method according to claim 1, wherein calculating an index includes
normalizing the power spectral density of the range of low frequencies,
normalizing the power spectral density of the range of high frequencies, and
setting the index equal to the normalized power spectral density of the range of high frequencies divided by the normalized power spectral density of the range of low frequencies.

7. The method according to claim 1, further comprising:
adjusting the R-R intervals to be equidistant using spline interpolation, and
resampling at a frequency of 5.0 HZ.

8. The method according to claim 1, wherein calculating R-R interval power spectra includes performing a fast Fourier transform on the electrocardiogram.

9. The method according to claim 1, wherein outputting the index includes
displaying the index for at least one individual, and
creating a graph consisting of the current index and a plurality of past indexes for the individual.

10. A method comprising:
receiving systolic arterial pressure and diastolic arterial pressure from at least one individual,
obtaining a pulse pressure based on the received systolic arterial pressure and the received diastolic arterial pressure, and
outputting the pulse pressure.

11. The method according to claim 10, further comprising determining whether medical assistance is required for the individual based on the pulse pressure.

12. A method comprising:
receiving vital sign information including arterial pressures and electrocardiogram from a plurality of remote individuals;
obtaining a pulse pressure for each individual;
detecting R-waves in received electrocardiograms;
determining R-R intervals in the electrocardiogram for each individual;
when three or more sampling periods for an individual the systolic pressure is progressively increasing or decreasing systolic pressures and the R-R intervals are progressively increasing or decreasing, calculating a baroreflex sensitivity;
for each individual
performing a fast Fourier transform on the received electrocardiogram to obtain R-R interval power spectra,
calculating power spectral densities for a range of low frequencies and a range of high frequencies,
calculating an index equal to the power spectral density of the range of high frequencies divided by the power spectral density of the range of low frequencies; and
notifying an entity when at least one indicator selected from a group consisting of the index exceeding a predetermined value, the pulse pressure is lower than a predetermined value, and the baroreflex sensitivity is trending lower.

13. The method according to claim 12, further comprising providing medical assistance to the individual who prompted the notification.

14. The method according to claim 13, further comprising prioritizing medical assistance for the individual based on the indicator as compared to indicators for other individuals awaiting medical assistance.

15. The method according to claim 12, further comprising displaying information regarding at least one indicator associated with at least one individual.

16. The method according to claim 12, further comprising displaying vital sign information, R-R interval power spectra, electrocardiogram waveform, graph displaying the current index and a plurality of previous indexes associated with at least one individual.

17. The method according to claim 12, wherein notifying an entity includes notifying a first responder to provide medical assistance to the individual.

18. The method according to claim 12, further comprising:
establishing a communication link with vital sign monitoring equipment on at least one remote individual to obtain vital sign information, and
terminating the communication link after sufficient vital sign information is obtained for processing.
